(19) United States
(12) Patent Application Publication
(10) Pub. No.: US 2006/0099267 A1
(43) Pub. Date: May 11, 2006

(54) High-dosage extended-release formulation of gepirone

(75) Inventors: Stephen J. Kramer, Houston, TX (US); Louis F. Fabre, Houston, TX (US)

Correspondence Address: OBLON, SPIVAK, MCCLELLAND, MAIER & NEUSTADT, P.C., 1940 DUKE STREET, ALEXANDRIA, VA, 22314, US

(73) Assignee: Fabre-Kramer Pharmaceuticals, Inc., Houston, TX (US)

(21) Appl. No.: 10/981,663

(22) Filed: Nov. 5, 2004

(51) Int. Cl. A61K 31/506; A61K 9/14

(52) U.S. Cl. 424/488; 514/252.18

(57) ABSTRACT

The present invention relates to high-dosage extended-release formulation of gepirone and methods of treating major depression by administering the same to a subject in need thereof. More specifically, the present invention relates to a high-dosage extended-release tablet form of gepirone. The present invention also relates to a method of treating depression in mammals by administering to a subject in need thereof an effective amount the high-dosage extended-release formulation of gepirone in accordance with the present invention.

### BACKGROUND OF THE INVENTION

1. Field of the Invention

The present invention relates to high-dosage extended-release formulation of gepirone and methods of treating major depression by administering the same to a subject in need thereof. More specifically, the present invention relates to a high-dosage extended-release tablet form of gepirone. The present invention also relates to a method of treating depression in mammals by administering to a subject in need thereof an effective amount the high-dosage extended-release formulation of gepirone in accordance with the present invention.

2. Discussion of the Background

Gepirone (also known as 4,4-dimethyl-1-[4-[4-(2-pyrimidinyl)-1-piperazinyl]-butyl]-2,6-piperidinedione hydrochloride) can be obtained by the process described in Example 7 of Temple, U.S. Pat. No. 4,423,049, (which is incorporated herein in its entirety by reference) and has the following structure:

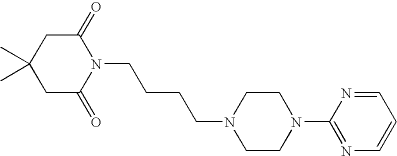

Gepirone and its salts are known to possess antidepressant and anxiolytic properties, presumably by serving as an agonist of the 5-HT_{1A} receptor. They are typically used to treat depression, dysthymia, impulse disorders, panic attacks and the like. However, in immediate-release formulations, gepirone has a short half-life. The time to maximum drug concentration of gepirone in the bloodstream (Tₘₐₓ) is about 1 hour and its T₅₀ (i.e., time until 50% of the drug has been released under controlled in vitro conditions) is about 2.5-3 hours (see U.S. Pat. No. 5,478,572, incorporated herein in its entirety by reference).

Owing to its rapid metabolism, gepirone has been administered in the past in several small dosages—e.g., 5 to 10 mg doses, 2 to 3 times per day. However, this multiple dosing scheme can lead to compliance problems. Moreover, failure to take the second or third dose results in unacceptably low plasma levels of gepirone. Further, studies indicate that, for 15 to 20 hours after administration, oral immediate release gepirone formulations can yield significant variations in human plasma concentrations. (For as further discussion see D. S. Robinson et al, Clinical Therapeutics, 1618-1633 (2003)).

However, as with any medicinal compound, each patient requires a different dosage level to specifically tailor the therapy to that patient's physical make-up, age, and disease complexity. Therefore, even with the low-dosage extended-release form of gepirone disclosed in U.S. Pat. No. 5,478,572, multiple dosing may be required (i.e., 4 tablets of a 20-mg dosage as opposed to a single high dosage tablet), which is inconvenient and expensive. And, as such, there remains a critical need for high-dosage extended-release form of gepirone having a dissolution profile analogous to the low-dosage extended-release form of gepirone disclosed in U.S. Pat. No. 5,478,572, while maintaining the same relative bioequivalence.

Heretofore, it was believed that it was not possible to formulate a high-dosage extended-release form of gepirone that would not result in an unsatisfactory increase in the amount of 1-(2-pyrimidinyl)piperazine (gepirone's principal metabolite, which is believed to be responsible for adverse side effects, including dizziness, nausea, headache and drowsiness) or a decrease in the relative efficacy. This skepticism was borne of the fact that in order to sufficiently control the release of gepirone the binder content would have to be increased to such a degree such that the resultant tablet for oral administration would end up being a “horse pill” (i.e., prohibitively large). Alternatively, the dosage administration would have to be formulated for delivery by a less desirable means (e.g., intravenous administration).

### SUMMARY OF THE INVENTION

It is an object of the present invention to provide a pharmaceutical composition, containing:
(1) from about 14 to about 25 wt % gepirone, or bioactive metabolite thereof, as a free base or a pharmaceutically acceptable salt thereof;
(2) from about 70 to about 85 wt % of a pharmaceutically acceptable cellulosic polymer matrix; and
(3) suitable amounts of one or more pharmaceutically acceptable excipients, wherein the release rate of gepirone from the dosage form is such that about 18 to 24 hours are required to attain from about 90 to about 95% absorption of gepirone.

In this object of he invention, (3) further comprises at least one pharmaceutically acceptable excipient selected from the group consisting of colorant, microcrystalline cellulose, colloidal silica and magnesium stearate; (2) is hydroxypropylmethylcellulose having a viscosity of from about 15,000 cps to about 100,000 cps; and (a) ranges from about 15.5 to about 18.7 wt %.

Another object of the present invention is to provide a composition containing:
(a) from about 14.0 to about 24.4 wt % gepirone, or bioactive metabolite thereof, as a free base or a pharmaceutically acceptable salt thereof,
(b) from 70.5 to 82.1 wt % hydroxypropylmethylcellulose having a viscosity of from about 15,000 to about 100,000 cps.,
(c) 0 to about 1 wt % colorant,
(d) about 8.0 to about 16.7 wt % microcrystalline cellulose,
(e) about 0.39 to about 0.47 wt % colloidal silica, and
(f) about 0.29 to about 1.0 wt % magnesium stearate.

In another object of the present invention is to provide a composition containing:
(a) about 15.6 wt % of gepirone hydrochloride
(b) about 75.3 wt % hydroxypropylmethylcellulose,
(c) about 0.31 wt % yellow ferric oxide,
(d) about 8.0 wt % microcrystalline cellulose,
(e) about 0.42 wt % colloidal silica, and
(f) about 0.31 wt % magnesium stearate.

In another object of the present invention is to provide a composition containing:
(a) about 19.5 wt % of gepirone hydrochloride
(b) about 70.7 wt % hydroxypropylmethylcellulose,
(c-1) about 0.24 wt % yellow ferric oxide,
(c-2) about 0.61 wt % red ferrid oxide
(d) about 8.2 wt % microcrystalline cellulose,
(e) about 0.39 wt % colloidal silica, and
(f) about 0.29 wt % magnesium stearate.

In each of the aforementioned objects it is yet a further object of the present invention to provide a tablet form of the high-dosage extended-release forms of gepirone.

In a preferred object of the present invention, the tablet has an ovoid-rectangular shape with either biconvex or flat faces. In this object, the tablet has an overall dimension of 0.400±0.05 inches by 0.325±0.05 inches with a thickness of 0.240±0.025 inches (biconvex faces) or a thickness of 0.195±0.025 inches (flat faces). Also within this object, the total weight of said tablet preferably ranges from 350 to 450 mg.

In yet another object of the present invention is to provide a method of ameliorating depression, anxiety, or psychological disorders (in particular depression) by administering to a patient (in particular a human) in need thereof an effective amount of the compositions or tablet form of the compositions in accordance with the present invention.

Another object of the present invention is to provide methods of making the aforementioned tablet forms of the compositions of the present invention.

The above objects highlight certain aspects of the invention. Additional objects, aspects and embodiments of the invention are found in the following detailed description of the invention.

### BRIEF DESCRIPTION OF THE FIGURES

A more complete appreciation of the invention and many of the attendant advantages thereof will be readily obtained as the same becomes better understood by reference to the following Figures in conjunction with the detailed description below.

FIG. 1 shows the comparative dissolution profile of 20 mg and 60 mg tablets described in the Preparation Example under in 0.01 M HCl (see Example 1).

FIG. 2 shows the comparative dissolution profile of 20 mg and 60 mg tablets described in the Preparation Example under in water (see Example 1).

FIG. 3 shows the comparative dissolution profile of 20 mg and 60 mg tablets described in the Preparation Example under in a pH 4.5 media (see Example 1).

FIG. 4 shows the comparative dissolution profile of 20 mg and 60 mg tablets described in the Preparation Example under in a pH 6.8 media (see Example 1).

### DETAILED DESCRIPTION OF THE INVENTION

Unless specifically defined, all technical and scientific terms used herein have the same meaning as commonly understood by a skilled artisan in biochemistry, chemistry, pharmacology, and the medical sciences.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

The present invention is based, in part, on the inventor's discovery that a high-dosage extended-release form of gepirone can be obtained wherein the this dosage for has a comparable dissolution profile and bioequivalence (for 1-PP, 3′-OH gepirone, and gepirone) to that of the low-dosage extended-release form of gepirone disclosed in U.S. Pat. No. 5,478,572, despite a reduction in the overall ratio between active ingredients and the release-controlling polymer (binder).

Extended release drug forms (especially gepirone) offer several advantages over immediate-release systems. Patient compliance is better because the extended release dosage forms need be taken only once in a 24-hour period. Thus, plasma concentration levels do not vary unacceptably—i.e., give high initial drug levels that are associated with the incidence of unwanted side effects, as well as having rapid drops in drug levels to below therapeutic levels—when the ER dosage forms are administered. In addition, in the case of gepirone, due to the slow time-release 1-PP levels are maintained at a satisfactory level thus avoiding significant complications arising from this metabolite.

In a general embodiment, the present invention provides a pharmaceutical composition for making an oral extended release gepirone dosage form comprising:

(1) from about 14 to about 25 wt % gepirone, or bioactive metabolite thereof, as a free base or a pharmaceutically acceptable salt thereof;

(2) from about 70 to about 85 wt % of a pharmaceutically acceptable cellulosic polymer matrix; and

(3) suitable amounts of one or more pharmaceutically acceptable excipients, wherein the release rate of gepirone from the dosage form is such that about 18 to 24 hours are required to attain from about 90 to about 95% absorption of gepirone.

In a particularly preferred embodiment of the present invention is an extended-release oral dosage form of gepirone, containing, expressed in weight percent:

(a) about 14.0 to about 24.4% (preferably, about 15.5 to about 18.7%) gepirone, or bioactive metabolite thereof, as a free base or a pharmaceutically acceptable salt thereof, for example a hydrochloride salt,

(b) about 70.5 to about 82.1% hydroxypropylmethylcellulose having a viscosity of from about 15,000 to about 100,000 cps.,

(c) 0 to about 1% (preferably 0 to about 0.3%) iron oxide,

(d) about 8.0 to about 16.7% microcrystalline cellulose,

(e) about 0.39 to about 0.47% (preferably about 0.42 to about 0.47%) colloidal silica, and

(f) about 0.29 to about 1.0% magnesium stearate.

Although the aforementioned embodiment is defined in terms of particular compounds for each component, it is to be understood that within the scope of the present invention these components may be replaced individually or in various combinations as described below. For each of the components below it is to be understood that the recited weight percentage reflects the total concentration of each component. For example, if there is a mixture of compounds that fall within the scope of a defined component group the recited weight percentage reflects the total for that mixture of compounds.

Component (a):

In a preferred embodiment, component (a) is present in the extended-release oral dosage form in a weight percentage of about 14.0 to about 24.4%, more preferably about 15.5 to about 18.7%. It is particularly preferred that component (a) be gepirone or a pharmaceutically acceptable salt thereof.

In vivo, gepirone is metabolized resulting in to major pharmacologically active metabolites: 1-(2-pyrimidinyl)piperazine (1-PP) and 3′-OH gepirone (D. S. Robinson et al, Clinical Therapeutics, pp. 1618-1633 (2003)). Two additional metabolites that may also posses bioactivity are also formed in vivo: 5-OH gepirone and 3′,5-dihydroxy gepirone.

In humans, the release of 1-PP, a common azapirone metabolite, is believed to be responsible for adverse side effects, including dizziness, nausea, headache and drowsiness. Moreover, 1-PP is a presynaptic α-2-adrenoceptor antagonist and it has been reported that it did not exhibit antidepressant-like characteristics in pre-clinical tests (D. S. Robinson et al, Clinical Therapeutics, pp. 1618-1633 (2003)). In contrast, the bioactive metabolites (3′-OH gepirone, in particular) have significant affinity for 5-HT_{1A} receptors. 3′-OH gepirone has been demonstrated to modify 5-HT neurotransmission in a comparable manner to gepirone even though 3′-OH has been found to exhibit full agonism at post-synaptic receptors in the hippocampus, whereas gepirone is a partial agonist (D. S. Robinson et al, Clinical Therapeutics, pp. 1618-1633 (2003)).

In view of the foregoing, in an embodiment of the present invention one or more of the bioactive gepirone metabolites 3′-OH gepirone, 5-OH gepirone, and 3′,5-dihydroxy gepirone, or a pharmaceutically acceptable salt thereof, may be used in place of gepirone.

Further, although the pharmaceutically acceptable salt form is preferred, it is contemplated that gepirone or the bioactive gepirone metabolites may be in a hydrate form, an enantiomeric form or mixture, or crystal form.

The pharmaceutical compounds suitable for administration in the present invention may be hydrochloride salts, but the free bases and other pharmaceutically acceptable salts are also suitable. The term “pharmaceutically acceptable salt” is well known in the art and the artisan is directed to S. M. Berge, et al. (J. Pharmaceutical Sciences, 66: 1-19 (1977), incorporated herein by reference) for a further description. Suitable pharmaceutically acceptable salts for administration in the present invention include acid addition salts. The acid addition salt may be formed by mixing a solution of the compound with a solution of a pharmaceutically acceptable non-toxic acid such as hydrochloric acid, hydrobromic acid, fumaric acid, maleic acid, succinic acid, acetic acid, citric acid, tartaric acid, carbonic acid, phosphoric acid, perchloric acid, sulphuric acid, oxalic acid, or malonic acid. Where the compound carries an acidic group, for example a carboxylic acid group, the present invention also contemplates salts thereof, preferably non-toxic pharmaceutically acceptable salts thereof, such as the sodium, potassium and calcium salts thereof.

Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pictate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, salts of amine groups. Salts of amine groups may also comprise the quaternary ammonium salts in which the amino nitrogen atom carries an alkyl, alkenyl, alkynyl or aralkyl group, nontoxic ammonium, quaternary ammonium, and amine cations formed using counterions such as halide, hydroxide, carboxylate, sulfate, phosphate, nitrate, loweralkyl sulfonate and aryl sulfonate.

Component (b):

In a preferred embodiment, component (b) is present in the extended-release oral dosage form in a weight percentage of about 70.5 to about 82.1%. Preferably, component (b) is hydroxypropylmethylcellulose having a viscosity of from about 15,000 to about 100,000 cps, which is a release-controlling polymer & binder (a.k.a., sustaining agent).

For oral formulations and dosage forms, the use of a polymeric cellulose matrix, or sustaining agent, is preferred. Suitable matrixes include hydroxyalkylsubstituted alkylcelluloses having viscosities of about 15,000 cps to about 100,000 cps. Examples of acceptable hydroxymethyl propylcellulose (HPMC) samples include grades K15M and K100M (i.e., 15,000 and 100,000 cps, respectively).

HPMC may be replaced (all or in part) or added to in the present invention, the replacement of some or the entire HPMC matrix may be with dicalcium phosphate or lactose, each of which generally increases dissolution rates.

In addition to the binder of component (b), the formulation of the present invention may also contain auxiliary binding agents, such as syrup, acacia, gelatin, sorbitol, tragacanth, or polyvinyl pyrrolidone. Examples of suitable fillers or extenders also include starches (e.g., maize-starch), lactose, sorbitol, glycine, sucrose, glucose, mannitol, and silicic acid, sodium citrate and dicalcium phosphate. Examples of other suitable binders may include chitosan, alginates, gelatin, polyvinylpyrrolidinone, sucrose, acacia, and mixtures thereof.

In an embodiment of the present invention the ratio of gepirone to binder (gepirone:binder) ranges from 1:3.5 to 1:14.5. Preferably, the binder is hydroxymethyl propylcellulose (HPMC).

Component (c):

In a preferred embodiment, component (c) is present in the extended-release oral dosage form in a weight percentage of 0 to about 1%, preferably 0 to about 0.3%. Although component (c) may be any colorant, it is preferred that the colorant be an iron oxide. In a particularly preferred embodiment the iron oxide is red ferric oxide, yellow ferric oxide, or mixtures thereof.

Additional exemplary colorants that may be used in place of or in addition to the foregoing include, but are not limited to, FD&C and D&C lakes, titanium dioxide, iron oxides, natural pigments, or dyes approved for ingestion by the U.S. Federal Drug Administration, or combinations thereof.

Component (d):

In a preferred embodiment, component (d) is present in the extended-release oral dosage form in a weight percentage of about 8.0 to about 16.7%. Although component (d) may be any diluent and/or compression aid, it is preferred that the diluent/compression aid be microcrystalline cellulose.

Additional diluents may include, but are not limited to, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

In yet another embodiment of the present invention, the ratio of gepirone to diluent (gepirone:diluent) ranges from 1:3 to 2.5:1. Preferably, the diluent is microcrystalline cellulose.

Component (e):

In a preferred embodiment, component (e) is present in the extended-release oral dosage form in a weight percentage of about 0.39 to about 0.47%, preferably about 0.42 to about 0.47%. Although component (e) may be any glidant, it is preferred that the glidant be colloidal silica (colloidal silicon dioxide).

Additional glidants include, but are not limited to, cornstarch, talc, or stearic acid, or combinations thereof.

Component (f):

In a preferred embodiment, component (f) is present in the extended-release oral dosage form in a weight percentage of about 0.29 to about 1.0%. Although component (f) may be any lubricant, it is preferred that the lubricant be magnesium stearate.

Additional lubricants may include, but are not limited to cellulose, talc, polyethyleneglycol, silicas, sodium lauryl sulfate, calcium stearate, and mixtures thereof.

Alternative Components:

It is contemplated in the present invention that the extended-release oral dosage form may also contain, in addition to components (a) through (f), various additional components. Based on the description above and Remington's Pharmaceuticals Sciences, 18th Edition (incorporated herein by reference), especially Part 8 therein, “Pharmaceutical Preparations and Their Manufacture, it would be well within the purview of the skilled artisan to add new compounds to the preferred embodiments described above.

In an embodiment of the present invention, the extended-release oral dosage form may include one or more additional pharmaceutically acceptable carriers. As used herein, the term “pharmaceutically acceptable carrier” means a non-toxic, inert solid, semi-solid or liquid filer, diluent, encapsulating material or formulation auxiliary. Some examples of materials which can serve as pharmaceutically acceptable carriers are already mentioned above, therefore, it is understood that in this aspect of the invention, pharmaceutically acceptable carriers may be any type of filer, diluent, encapsulating material or formulation auxiliary not previously mentioned.

In another embodiment, the extended-release oral dosage form may include may also include one or more releasing agents, coating agents, sweetening, flavoring and perfuming agents, preservatives and antioxidants.

The flavorant(s), which is used primarily for taste- and/or odor-masking, may be vanillin, sodium citrate, citric acid, mint, orange, lemon oil, or any other pharmaceutically approved flavorant or tastemasking agent, and combinations thereof.

Shape And Physical State Of The High-Dosage Extended-Release Form Of Gepirone

The present invention contemplates extended-release oral dosage forms that are suitable for orally administration. These forms include tablets, capsules, caplets, lozenges, powders, suspensions, syrups and the like are suitable forms. Preferentially, the tablet may be mentioned including those having a convex shape, a spherical (i.e., round) shape, or a capsule shape.

In a preferred embodiment, the extended-release oral dosage form of gepirone is a tablet. Preferably, the tablet has oval shape, which enhances their surface area and improves the release of gepirone therefrom. More preferably, the tablet has an ovoid-rectangular shape, which may have either flat or biconvex faces.

When the extended-release oral dosage form of gepirone is a tablet having an ovoid-rectangular shape, it is preferred that the tablets have an overall dimension of 0.400±0.05 inches by 0.325±0.05 inches with a thickness of 0.240±0.025 inches (biconvex faces) or 0.195±0.025 inches (flat faces).

In another embodiment of the present invention the overall tablet weight ranges from 350 to 450 mg, preferably from 375 to 425 mg, more preferably from 385 to 410 mg. Further, within the context of the present invention, the tablet weight range of 350 to 450 mg, preferably from 375 to 425 mg, more preferably from 385 to 410 mg correspond to the total weight of the unit dose form.

Therefore, the pharmaceutical compositions (i.e., extended-release oral dosage form of gepirone) of the present invention preferably contain between about 55 to 100 mg (preferably from 60 to 80 mg, more preferably 60 or 80 mg) of the active ingredient per unit dose.

The tablets may, if desired, be coated using known methods and excipients that may include enteric coating using for example hydroxypropylmethylcellulose phthalate. Such tablets may, if desired, be provided with enteric coatings by known methods, for example by the use of cellulose acetate phthalate. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Similarly, capsules, for example hard or soft gelatin capsules, containing the active compound with or without added excipients, may be prepared by known methods and, if desired, provided with enteric coatings in a known manner. The contents of the capsule may be formulated using known methods so as to give sustained release of the active compound. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Solid compositions of a similar type may also be employed as fillers in soft and hard-filled gelatin capsules using such excipients as lactose or milk sugar as well as high molecular weight polyethylene glycols and the like.

Treatment Methods:

Gepirone can be orally administered in once-a-day extended release dosage forms, which contain gepirone hydrochloride (or a bioactive metabolite thereof), a cellulosic polymer matrix and suitable amounts of pharmaceutical excipients. The resultant gepirone formulation yields oral products the take about 19 to about 24 hours to release 90 to 95% of the active agent.

Therefore, in yet another embodiment of the present invention is a method of treating a patient diagnosed as suffering from major (primary or general) depression, with or without generalized anxiety disorder, comprising administering to said patient an effective amount of the extended-release oral dosage form gepirone as described in the present application.

In this embodiment, the patient (preferably a human, but inclusive of all mammals) in need of treatment is first diagnosed as suffering from a particular depressive condition. For a discussion of patient evaluation, diagnostic criteria, etc. of anxiety and depression, the skilled artisan is referred to: DSM-IV-TR (Diagnostic and Statistical Manual of Mental Disorders, American Psychiatric Association, Washington, D.C., 2000; Depressive Disorders: 369-428, and Anxiety Disorders: 429-484).

To this end, the gepirone extended-release composition and dosage forms of the invention are designed to deliver an effective anti-depressive and/or anxiolytic amount of gepirone or a pharmaceutically acceptable salt thereof to a mammal, preferably a human patient.

“Effective” doses as used herein refer to an amount of about 0.01 to 40 mg/kg body weight/day are contemplated, preferably 0.05 to 20 mg/kg body weight/day, more preferably 0.1 to 2 mg/kg of body weight/day. For certain central nervous system disorders, 15 to 90 mg/day, preferably 30-60 mg/day, are recommended. See U.S. Pat. No. 4,771,053 and U.S. Pat. No. 5,478,572. Alternatively, the effective dose or delivery system should result in plasma concentrations in the range of about 1 ng/ml to about 20 ng/ml, preferably about 1 ng/ml to about 5 ng/ml.

As described above, in an embodiment of the present invention, the extended-release oral dosage form of gepirone for use in the inventive method contains, expressed in weight percent:

(a) about 14.0 to about 24.4% (preferably, about 15.5 to about 18.7%) gepirone, or bioactive metabolite thereof, as a free base or a pharmaceutically acceptable salt thereof, for example a hydrochloride salt,

(b) about 70.5 to about 82.1% hydroxypropylmethylcellulose having a viscosity of from about 15,000 to about 100,000 cps.,

(c) 0 to about 1% (preferably 0 to about 0.3%) iron oxide,

(d) about 8.0 to about 16.7% microcrystalline cellulose,

(e) about 0.39 to about 0.47% (preferably about 0.42 to about 0.47) colloidal silica, and

(f) about 0.29 to about 1.0% magnesium stearate.

As stated herein above, gepirone in the extended-release oral dosage form of gepirone also embraces the bioactive gepirone metabolites selected from the group consisting of 3′-OH gepirone, 3′,5-dihydroxy gepirone, and 5-OH gepirone. The method may employ any one of these compounds. However, combinations of these metabolites, or combinations of the metabolites with other active or inert ingredients, are also contemplated.

The present invention further provides methods for ameliorating depression, anxiety, or psychological disorders in a patient (preferably human, but inclusive of all mammals) in need of such treatment, by administering to the patient an effective amount or dose of gepirone or a bioactive gepirone metabolite such as 3′-OH gepirone, 3′,5-dihydroxy gepirone, and 5-OH gepirone. As used herein, the administration of gepirone or a bioactive gepirone metabolite includes the administration of any active salt form, hydrate form, enantiomeric form or mixture, or crystal form of the compound.

Also within the scope of the present invention are methods in which the extended-release oral dosage form of gepirone is administered (sequentially or simultaneously) with one or more anti-depressive or anxiolytic agents in a combination therapy for treatment of depression and/or anxiety.

Production Methods:

The extended-release oral dosage form of gepirone of the present invention may be made by the following procedure: (i) admixing all or a part of components (a) through (f), (ii) blending the mixture, (iii) adding a part of or the remainder (if any) of components (a) through (f), (iv) blending the mixture (if remainder added during (iii)), (v) slugging, (vi) milling, (vii) adding the remainder of the lubricant (component (f); if part reserved during (i) and (iv)), (viii) blending the mixture; (ix) compressing the final finished tablet blend.

Of course it is to be understood that the aforementioned steps may be rearranged or substituted so long as the rearrangement or substitution does not substantially alter the pharmacological efficacy of the resultant dosage form from the process described above. In this regard, the artisan is referred to Remington's Pharmaceuticals Sciences, 18th Edition (incorporated herein by reference), for example Part 8 therein, “Pharmaceutical Preparations and Their Manufacture.

Further, the artisan is directed to the processing procedures described in U.S. Pat. Nos. 4,423,049 and 5,478,572 (incorporated herein by reference) as providing relevant examples of the extended-release oral dosage form of gepirone production process described above.

In a preferred embodiment, the following procedure is followed to make the inventive extended-release oral dosage form of gepirone:

i) admixing all of component (a), all of component (c), all of component (e), and 20% of the total concentration of component (b);

ii) blending the mixture from (i);

iii) adding to the blend from (ii) all of component (d), half of the total concentration of component (f), and the remaining 80% of the total concentration of component (b);

iv) blending the mixture from (iii);

v) slugging the blend from (iv);

vi) milling the slug from (v);

vii) adding the remaining 50% of component (f) to the milled slug from (vi);

viii) blending the mixture from (vii);

ix) compressing the blend from (viii) into the final desired form.

Typically, the resultant blends are compressed in step (ix) into tablets or into micropellets. If micropellets are made, they are optionally overcoated with conventional coating adjuvant(s) and then tableted or filled into capsules.

In the case where an enteric coating is desired, this coating would typically be added following step (ix).

It is to be understood that each of the above-described manufacturing processes may be scaled-up to an industrially applicable scale and is in no way limited to individual dosage preparation methods. It is also contemplated that alterations to each step (individually or collectively) may be made to facilitate the production of the inventive extended-release oral-dosage form of gepirone, so long the modifications do not alter the composition of the final product beyond the tolerance limits described above or the bioefficacy of the resultant product.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

Where a numerical limit or range is stated herein, the endpoints are included. Also, all values and subranges within a numerical limit or range are specifically included as if explicitly written out. In this regard, where the term “about” is recited, this term is understood to include the recited value, as well as values that are within 1% of the recited value (either above or below depending upon whether “about” defines an upper or lower boundary), preferably with 0.5% of the recited value.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLES

### Preparation Example

Following the procedure described above and in U.S. Pat. No. 5,478,572, tablet forms of the extended-release oral dosage form of gepirone were prepared. Generally, this procedure entailed (i) admixing all of gepirone hydrochloride, all of the colorant, all of the colloidal silicon dioxide, and 20% of the total mass of hydroxypropylmethylcellulose; (ii) blending the mixture from (i) for 15 minutes and subsequently delumping using a Fitzmill #0020 plate; (iii) adding to the blend from (ii) all of microcrystalline cellulose, 50% of the total mass of magnesium stearate, and the remaining 80% of the total mass of hydroxypropylmethylcellulose; (iv) blending the mixture from (iii) for 28 minutes; (v) slugging the blend from (iv) using a rotary tablet press; (vi) milling the slug from (v) using a Fitzmill #0093 plate; (vii) adding the remaining 50% of magnesium stearate to the milled slug from (vi); (viii) lubricating/blending the mixture from (vii) for 7 minutes; and (ix) compressing the blend from (viii) into the final desired tablet form.

In this manner ovoid-rectangular shaped tablets having biconvex faces were obtained. The overall dimensions of the tablets were 0.405 inches (102.87 mm) by 0.338 inches (85.85 m) with a cup depth of 0.050 inches (12.70 mm). The tablet weights and component quantity/unit are given in Table 1 below:

TABLE 1

| | | Quantity per unit (units in mg) | | | |
|---|---|---|---|---|---|
| | | 20 mg | 40 mg | 60 mg | 80 mg |
| Ingredients | Function | tablets | tablets | tablets | tablets |
| Gepirone HCl | Active | 20.0 (18.1)¹ | 40.0 (36.2)¹ | 60.0 (54.3)¹ | 80.0 (72.4)¹ |
| | ingredient | | | | |
| Colloidal | Glidant | 1.6 | 1.6 | 1.6 | 1.6 |
| silicon dioxide | | | | | |
| Yellow ferric | Colorant | 0.4 | 0.08 | 1.2 | 1.0 |
| oxide | | | | | |
| Red ferric oxide | Colorant | | | | 2.5 |
| Hydroxypropyl | Release | 290.0 | 290.0 | 290.0 | 290.0 |
| methylcellulose | controlling | | | | |
| | polymer & | | | | |
| | binder | | | | |
| Microcrystalline | Diluent & | 61.8 | 52.12 | 31.0 | 33.7 |
| cellulose | compression | | | | |
| | aid | | | | |
| Magnesium | Lubricant | 1.2 | 1.2 | 1.2 | 1.2 |
| stearate | | | | | |
| Total Tablet | | 375.0 | 385.0 | 385.0 | 410.0 |
| Weight | | | | | |

¹indicates the free base content

### Example 1

### Dissolution Profiles for the 60-mg Formulation vs. the 20-mg Formulation

Comparative dissolution profiles for 20 mg and 60 mg tablets in all SUPAC-MR dissolution media are presented in Table 2 below.

TABLE 2

| | | Time (Hours) | | | | | | | | | |
|---|---|---|---|---|---|---|---|---|---|---|---|
| Medium | Strength | 1 | 2 | 4 | 8 | 10 | 12 | 14 | 16 | 18 | 20 |
| 0.01 M HCl | 20 mg | 20.2 | 30.4 | 45.1 | 64.7 | 71.8 | 77.8 | 82.8 | 86.9 | 90.5 | 93.4 |
| 0.01 M HCl | 60 mg | 22.0 | 32.8 | 48.2 | 68.6 | 76.0 | 82.0 | 87.1 | 91.2 | 94.5 | 97.3 |
| water | 20 mg | 19.8 | 29.8 | 44.0 | 63.1 | 70.1 | 76.0 | 80.9 | 85.0 | 88.4 | 91.3 |
| water | 60 mg | 21.9 | 32.7 | 48.2 | 68.6 | 75.8 | 81.7 | 86.6 | 90.5 | 93.7 | 96.2 |
| pH = 4.5 | 20 mg | 17.7 | 26.6 | 39.5 | 57.2 | 63.8 | 69.4 | 74.3 | 78.6 | 82.3 | 85.6 |
| pH = 4.5 | 60 mg | 20.2 | 30.0 | 43.8 | 62.2 | 68.8 | 74.3 | 79.1 | 83.1 | 86.5 | 89.4 |
| pH = 6.8 | 20 mg | 15.9 | 24.1 | 36.4 | 53.7 | 60.5 | 66.4 | 71.5 | 76.1 | 80.2 | 83.8 |
| pH = 6.8 | 60 mg | 17.5 | 26.4 | 39.6 | 57.6 | 64.4 | 70.2 | 75.1 | 79.5 | 83.3 | 86.6 |

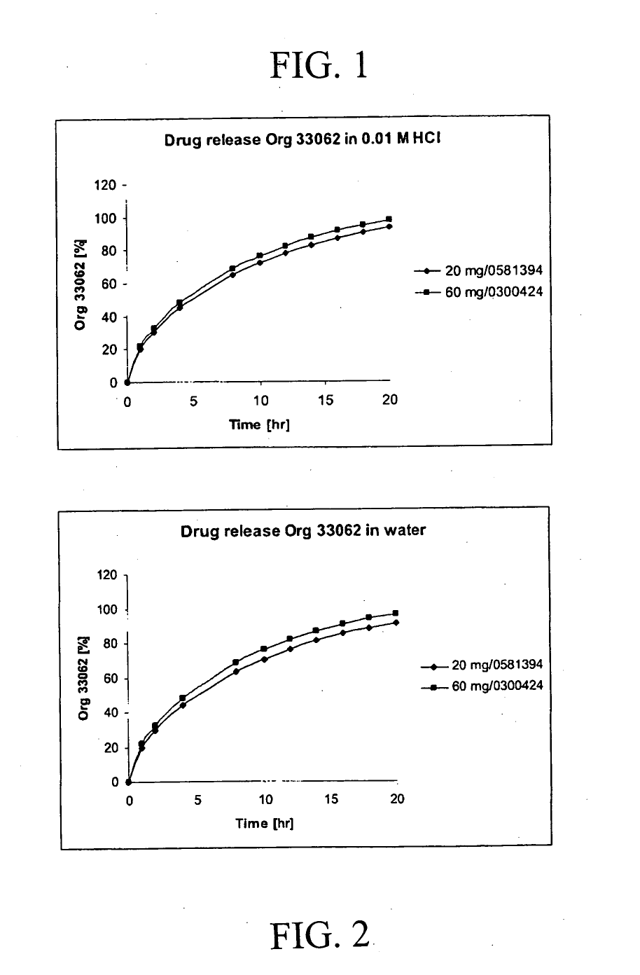

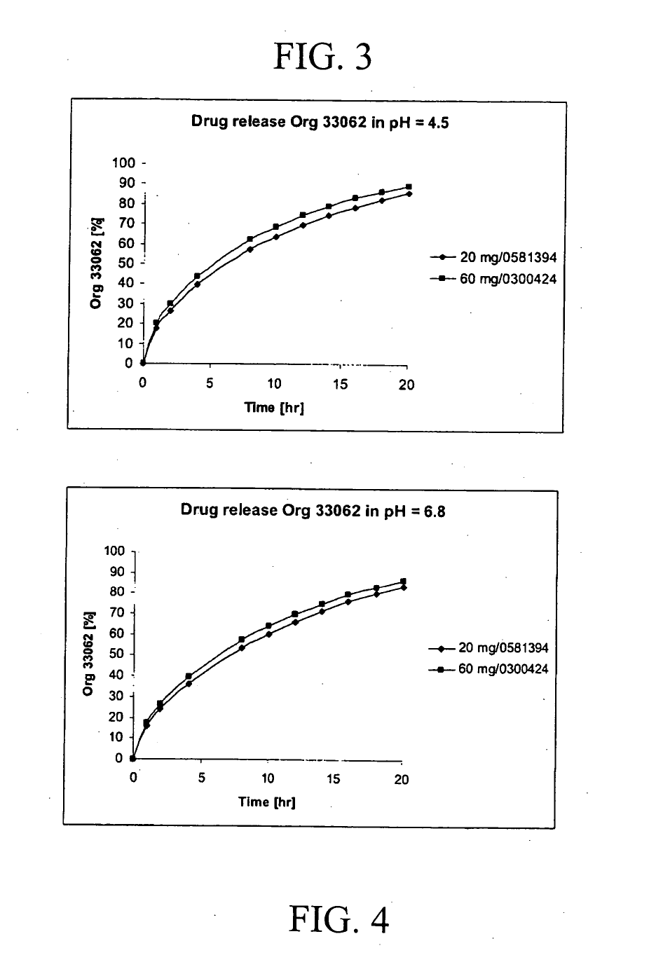

The results above are graphically represented in FIGS. 1-4.

The f1 and f2 calculation (see Table 3) for the comparative dissolution profiles demonstrates equivalence between the tablets in all 4 media

TABLE 3

| Medium | F1¹ | F2¹ |
|---|---|---|
| 0.01 M HCl | 5.2 | 71.2 |
| Water | 6.7 | 66.1 |
| pH = 4.5 | 6.8 | 70.1 |
| pH = 6.8 | 5.3 | 76.3 |
| Criteria² | 0-15 | 50-100 |

¹F1 and F2 provide insight and/or a measure of the distribution of results among batches.
²The criteria corresponds to acceptable F1 and F2 value ranges.

### Example 2

### Bioequivalence of the 80-mg Formulation vs. the 20-mg Formulation

Heretofore, efficacy and pharmacokinetic data for extended release gepirone tablets have been primarily obtained by administering one or multiple 20-mg extended-release gepirone tablets (similar to that described in the Preparation Example above). Therefore, studies were undertaken to demonstrate the bioequivalence of 40-mg (data not shown) and 80-mg extended-release gepirone tablets (as described in the Preparation Example above) versus multiple 20-mg extended-release gepirone tablets.

To this end, an open label, randomized, four-way crossover, single dose study design was used with a washout phase of one week between different treatments. During each study period, the subjects were hospitalized from the afternoon before until 36 hours after gepirone administration. Following discharge, blood sample collections were taken at 48, 60, and 72 hours post-administration. During the study period, gepirone was administered to the thirty two (32) subjects (average age 30.9±6.3 years; average weight 73.8±8.3 kg; average height 178.1±5.6; and average BMI 23.3±2.3 kg/m²) as a single oral dose of 40 mg or 80 mg. To this end, the following treatments were given in a randomized order (tablet formulation shown in the Preparation Example above):

A) 1×40-mg extended-release gepirone tablet (data not shown);

B) 2×20-mg extended-release gepirone tablets (data not shown);

C) 1×80-mg extended-release gepirone tablet;

D) 4×20-mg extended-release gepirone tablets.

After each treatment, blood samples were obtained from the subjects at regular intervals for 72 hours for determining the pharmacokinetics of gepirone, 1-PP, and 3′-OH gepirone.

To determine the bioequivalence of one 80-mg extended-release gepirone tablet versus four 20-mg extended-release gepirone tablets the following pharmacokinetic parameter were calculated for each subject and treatment:
tₘₐₓ (TMAX)
The actual time of occurrence of Cₘₐₓ.
Cₘₐₓ (CMAX)
The measured maximum concentration in plasma after single dosing.
dn-Cₘₐₓ (DCMAX)
The dose-normalized Cₘₐₓ was calculated as Cₘₐₓ/Dose.
λ_{Z} (LZ) and t_{1/2} (THALF)
The slope (β) of the terminal log-linear phase of the concentration-versus-time curve was determined by linear regression. The data had to be fit to the function:
   log₀Cᵢ=log₀C_{interc}−βtᵢ
starting with the last 3 (Cᵢ, tᵢ) data pairs for which Cᵢ≧LOQ; C_{interc} is the intercept with the concentration axis at zero time. The procedure continued, adding preceding data points one at a time and fitting the regression equation, until Cₘₐₓ occurring at the tₘₐₓ was reached. The terminal log-linear portion is then defined by the data yielding the smallest mean square error (MSE) term in the regression analysis. The terminal elimination rate constant (λ_{Z}) was defined as −β from which the elimination half-life (t_{1/2}) was calculated as log⁰2/λ_{Z}. Concentrations lower than LOQ in the elimination phase were ignored.
AUC₀₋ₜₗₐₛₜ (AUCT)
The AUC from zero to tₗₐₛₜ (AUC₀₋ₜₗₐₛₜ) was calculated by means of the linear trapezoidal rule, where tₗₐₛₜ represented the last time point with a measurable concentration within a subject.
dn-AUC₀₋ₜₗₐₛₜ (DUCT)
The dose-normalized AUC₀₋ₜₗₐₛₜ was calculated as AUC₀₋ₜₗₐₛₜ/Dose.
AUC_{0-∞} (AUC)
The AUC from zero to infinity was calculated as AUC_{0-∞}=AUC₀₋ₜₗₐₛₜ+AUC_{tlast-∞}. For AUC₀₋ₜₗₐₛₜ see above ad AUC_{tlast-∞}=Cₜₗₐₛₜ/λ_{Z}, where Cₜₗₐₛₜ was the fitted concentration at time tₗₐₛₜ using the regression line for which λ_{Z} had been calculated.
dn-AUC_{0-∞} (DAUC)
The dose-normalized AUC_{0-∞} was calculated as AUC_{0-∞}/Dose.
CLₐₚₚ (CLAPP)
The true total plasma clearance (CL) after a single oral dose equals f•Dose/AUC_{0-∞} where f is the fractional absolute bioavailability of the oral preparation. Since f cannot be calculated from oral data alone, CL/F=Dose/AUC_{0-∞} was calculated and denoted as “apparent clearance” (CLₐₚₚ). The administered dose of gepirone was expressed as a free base. The amount free bases of gepirone was also used for CLₐₚₚ calculation of the 1-PP and 3′-OH gepirone metabolites.
wn-CLₐₚₚ (WCLAPP)
The weight-normalized CLₐₚₚ was calculated as CLₐₚₚ divided by the body weight (kg).
V_{z,app} (VZAPP)
The apparent volume of distribution during the terminal phase after a single oral dose was defined as V_{z,app}=CLₐₚₚ/λ_{Z}.
wn-V_{z,app} (WVZAPP)
The weight-normalized V_{z,app} was calculated as V_{z,app} divided by the body weight (kg).

For dn-Cₘₐₓ, dn-AUC_{0-∞}, and dn-AUC₀₋ₜₗₐₛₜ, 0.80-1.25 was used as acceptance range. The formulations were declared bioequivalent with respect to the tested parameters if the 90% confidence interval was fully contained within the acceptance range. This was done for gepirone, 1-PP and 3′-OH gepirone. For all other pharmacokinetic parameters, classical hypothesis testing was performed on the logo-transformed values using the same ANOVA models as for the bioequivalence testing.

The following mean (std. dev.) pharmacokinetic parameters were calculated for gepirone, 1-PP, and 3′-OH gepirone (see Table 4):

TABLE 4

| | | Gepirone (n = 32) | | | | 1-PP (n = 32) | | | | 3′-OH gepirone (n = 32) | | | |
|---|---|---|---|---|---|---|---|---|---|---|---|---|---|
| Parameter | Units | 1 × 80 mg | | 4 × 20 mg | | 1 × 80 mg | | 4 × 20 mg | | 1 × 80 mg | | 4 × 20 mg | |
| Cₘₐₓ | (ng/mL) | 11.2 | (5.36) | 10.2 | (7.05) | 14.2 | (10.0) | 13.1 | (7.05) | 26.9 | (7.24) | 25.1 | (6.99) |
| dn-Cₘₐₓ | (ng/mL/mg) | 0.140 | (0.067) | 0.128 | (0.088) | 0.178 | (0.125) | 0.164 | (0.088) | 0.337 | (0.091) | 0.314 | (0.087) |
| tₘₐₓ¹ | (h) | 5.5 | (2.0-12.0) | 6.0 | (0.5-12.0) | 5.0 | (3.0-18.0) | 6.0 | (2.0-12.0) | 6.0 | (4.0-12.0) | 8.0 | (4.0-12.0) |
| AUC₀₋ₜₗₐₛₜ | (ng · h/mL) | 151 | (69.2) | 149 | (89.8) | 250 | (214) | 260 | (200) | 551 | (170) | 557 | (181) |
| dn-AUC₀₋ₜₗₐₛₜ | (ng · h/mL/mg) | 1.89 | (0.865) | 1.86 | (1.12) | 3.12 | (2.67) | 3.25 | (2.50) | 6.89 | (2.13) | 6.96 | (2.27) |
| AUC₀₋ₒₒ | (ng · h/mL) | 158 | (73.4) | 155 | (88.1) | 259 | (213) | 268 | (202) | 558 | (170) | 562 | (183) |
| dn-AUC₀₋ₒₒ | (ng · h/mL/mg) | 1.98 | (0.917) | 1.94 | (1.10) | 3.24 | (2.66) | 3.35 | (2.52) | 6.97 | (2.13) | 7.03 | (2.29) |
| t_{1/2} | (h) | 7.93 | (8.26) | 8.22 | (9.12) | 9.43 | (6.06) | 7.41 | (3.34) | 8.14 | (3.30) | 7.40 | (2.13) |
| CLₐₚₚ | (L/h) | 692 | (804) | 625 | (386) | 417 | (249) | 396 | (242) | 146 | (61.7) | 144 | (53.9) |
| wn-CLₐₚₚ | (L/h/kg) | 9.70 | (12.7) | 8.61 | (5.95) | 5.73 | (3.78) | 5.41 | (3.54) | 2.01 | (0.971) | 1.98 | (0.836) |
| V_{z, app} | (L) | 6416 | (6156) | 7334 | (9108) | 5531 | (4982) | 4168 | (3289) | 1672 | (806) | 1437 | (346) |
| wn-V_{z, app} | (L/kg) | 86.1 | (80.8) | 102 | (137) | 74.0 | (62.1) | 57.0 | (44.7) | 22.8 | (11.0) | 19.8 | (5.92) |

¹median range

The statistical analyses concerning the average bioequivalence testing for gepirone, 1-PP, and 3′-OH gepirone are given in Table 5 below.

TABLE 5

| | | | | | |
|---|---|---|---|---|---|
| | Geom. mean test | Geom. mean reference | Point Estimate | 90% Confidence | |
| Parameter | formulation | formulation | test/ref. | interval | Conclusion |

Gepirone Comparison: 1 × 80 mg (test) versus 4 × 20 mg (reference)

| | | | | | |
|---|---|---|---|---|---|
| dn-Cₘₐₓ | 0.123 | 0.105 | 1.17 | 1.02-1.33 | Indeterminant |
| dn-AUC₀₋ₒₒ | 1.71 | 1.68 | 1.02 | 0.89-1.16 | Bioequivalent |
| dn-AUC₀₋ₜₗₐₛₜ | 1.63 | 1.59 | 1.03 | 0.90-1.18 | Bioequivalent |

1-PP Comparison: 1 × 80 mg (test) versus 4 × 20 mg (reference)

| | | | | | |
|---|---|---|---|---|---|
| dn-Cₘₐₓ | 0.151 | 0.146 | 1.03 | 0.94-1.13 | Indeterminant |
| dn-AUC₀₋ₒₒ | 2.60 | 2.74 | 0.95 | 0.87-1.04 | Bioequivalent |
| dn-AUC₀₋ₜₗₐₛₜ | 2.47 | 2.62 | 0.94 | 0.86-1.03 | Bioequivalent |

3′-OH Gepirone Comparison: 1 × 80 mg (test) versus 4 × 20 mg (reference)

| | | | | | |
|---|---|---|---|---|---|
| dn-Cₘₐₓ | 0.325 | 0.303 | 1.07 | 0.98-1.18 | Bioequivalent |
| dn-AUC₀₋₀₀ | 6.62 | 6.67 | 0.99 | 0.90-1.09 | Bioequivalent |
| dn-AUC₀₋ₜₗₐₛₜ | 6.54 | 6.61 | 0.99 | 0.90-1.09 | Bioequivalent |

As can be seen from Table 5, for all compounds the 1×80 mg tablet was found average bioequivalent to four 20 mg tablets with respect to dn-AUC. This is also the case for dn-Cₘₐₓ of 1-PP and 3′-OH gepirone. For gepirone an inconclusive result was found for dn-Cₘₐₓ.

Since the average bioequivalence testing leads to indeterminate conclusions in some cases for gepirone, despite the fact that the point estimate of the ratio of the geometric means of the test and reference treatments lies within the acceptance range 0.80-1.25. Therefore, an additional statistical analysis using the population approach as supportive evidence was performed. The results are shown in Table 6 where

S_{T}²: total sample variance (i.e. sum of within- and between-subject variances) of the test treatment based on loge 4ransformed parameters.

S_{R}²: total sample variance (i.e. sum of within- and between-subject variances) of the reference treatment based on loge transformed parameters.

S_{d}²: sample variance of the intra-subject differences.

θ₁, (0.50): the 50% quantile of the bioequivalence criterion of θ₁ (=median for θ₁).

θ₁ (0.95): the 95% quantile of the bioequivalence criterion of θ₁ (=upper bound of 95% confidence interval for θ₁).

Since for all analyses S_{R}²>σ_{T0}²(=0.2²=0.04), the reference-scaled method was applied.

TABLE 6

| | | | | | | |
|---|---|---|---|---|---|---|
| Parameter | S_{T}² | S_{R}² | S_{d}² | θ₁ (0.50) | θ₁ (0.95) | Conclusion*⁾ |

Gepirone Comparison: 1 × 80 mg (test) versus 4 × 20 mg (reference)

| | | | | | | |
|---|---|---|---|---|---|---|
| dn-Cₘₐₓ | 0.3318 | 0.3755 | 0.2699 | −0.033 | 0.401 | Bioequivalent |
| dn-AUC₀₋ₒₒ | 0.4063 | 0.3114 | 0.2747 | 0.268 | 0.876 | Bioequivalent |
| dn-AUC₀₋ₜₗₐₛₜ | 0.4154 | 0.3449 | 0.3233 | 0.193 | 0.768 | Bioequivalent |

1-PP Comparison: 1 × 80 mg (test) versus 4 × 20 mg (reference)

| | | | | | | |
|---|---|---|---|---|---|---|
| dn-Cₘₐₓ | 0.2467 | 0.2435 | 0.0763 | 0.018 | 0.269 | Bioequivalent |
| dn-AUC₀₋ₒₒ | 0.4128 | 0.4108 | 0.0883 | 0.007 | 0.253 | Bioequivalent |
| dn-AUC₀₋ₜₗₐₛₜ | 0.4338 | 0.4383 | 0.0935 | −0.007 | 0.251 | Bioequivalent |

3′-OH Gepirone Comparison: 1 × 80 mg (test) versus 4 × 20 mg (reference)

| | | | | | | |
|---|---|---|---|---|---|---|
| dn-Cₘₐₓ | 0.0684 | 0.0648 | 0.1069 | 0.108 | 0.632 | Bioequivalent |
| dn-AUC₀₋ₒₒ | 0.1132 | 0.1054 | 0.1333 | 0.078 | 0.710 | Bioequivalent |
| dn-AUC₀₋ₜₗₐₛₜ | 0.1137 | 0.1058 | 0.1360 | 0.077 | 0.714 | Bioequivalent |

*⁾Bioequivalent if θ₁, (0.95) ≦ θ_{P} (=1.745)

The results obtained with the population approach showed bioequivalence for Cₘₐₓ, AUC_{0-∞}, and AUC₀₋ₜₗₐₛₜ of gepirone, 1-PP and 3′-OH gepirone with respect to one 80-mg gepirone extended release tablet and four 20 mg gepirone extended-release tablets (the same was observed for one 40-mg gepirone extended release tablet and two 20 mg gepirone extended-release tablets). It was further determined that a comparison between the 40 mg tablet and the 80 mg tablet showed no statistically significant differences for gepirone or its metabolites. This finding indicates dose proportionality of gepirone in the 40-80 mg dose range.

Numerous modifications and variations on the present invention are possible in light of the above teachings. It is, therefore, to be understood that within the scope of the accompanying claims, the invention may be practiced otherwise than as specifically described herein.

1. A pharmaceutical composition, comprising:
(a) from about 14 to about 25 wt % gepirone, or bioactive metabolite thereof, as a free base or a pharmaceutically acceptable salt thereof;
(b) from about 70 to about 85 wt % of a pharmaceutically acceptable cellulosic polymer matrix; and
(c) suitable amounts of one or more pharmaceutically acceptable excipients, wherein the release rate of gepirone from the dosage form is such that about 18 to 24 hours are required to attain from about 90 to about 95% absorption of gepirone.

2. The composition of claim 1, wherein (c) further comprises at least one pharmaceutically acceptable excipient selected from the group consisting of colorant, microcrystalline cellulose, colloidal silica and magnesium stearate.

3. The composition of claim 1, wherein (b) is hydroxypropylmethylcellulose having a viscosity of from about 15,000 cps to about 100,000 cps.

4. The composition of claim 1, wherein the concentration of (a) ranges from about 15.5 to about 18.7 wt %.

5. The composition of claim 1, wherein (a) is gepirone or a pharmaceutically acceptable salt thereof.

6. The composition of claim 1, wherein (a) is a bioactive metabolite of gepirone or a pharmaceutically acceptable salt thereof.

7. The composition of claim 6, wherein said bioactive metabolite of gepirone is selected from the group consisting of 3′-OH gepirone, 3′,5-dihydroxy gepirone, and 5-OH gepirone.

8. A method of treating depression in a patient in need thereof, comprising administering to said patient an effective amount of the composition of claim 1.

9. The method of claim 8, wherein said patient is human.

10. The method of claim 8, wherein said patient further suffers from generalized anxiety.

11. The method of claim 8, wherein said effective amount ranges from 0.01 to 40 mg/kg body weight per day.

12. The composition of claim 1, wherein said composition is in a form of a tablet.

13. The composition of claim 12, wherein the total weight of said tablet ranges from 350 to 450 mg.

14. The composition of claim 13, wherein the weight of (a) in said table ranges from 60 to 80 mg.

15. The composition of claim 12, wherein said tablet has an ovoid-rectangular shape with biconvex faces.

16. The composition of claim 15, wherein said tablet has an overall dimension of 0.400±0.05 inches by 0.325±0.05 inches with a thickness of 0.240±0.025 inches.

17. The composition of claim 12, wherein said tablet has an ovoid-rectangular shape with flat faces.

18. The composition of claim 17, wherein said tablet has an overall dimension of 0.400±0.05 inches by 0.325±0.05 inches with a thickness of 0.195±0.025 inches.

19. A method of treating depression in a patient in need thereof, comprising administering to said patient an effective amount of the composition of claim 12.

20. The method of claim 19, wherein said patient is human.

21. The method of claim 19, wherein said patient further suffers from generalized anxiety.

22. The method of claim 19, wherein said effective amount ranges from 0.01 to 40 mg/kg body weight per day.

23. A composition comprising:
(a) from about 14.0 to about 24.4 wt % gepirone, or bioactive metabolite thereof, as a free base or a pharmaceutically acceptable salt thereof,
(b) from 70.5 to 82.1 wt % hydroxypropylmethylcellulose having a viscosity of from about 15,000 to about 100,000 cps.,
(c) 0 to about 1 wt % colorant,
(d) about 8.0 to about 16.7 wt % microcrystalline cellulose,
(e) about 0.39 to about 0.47 wt % colloidal silica, and
(f) about 0.29 to about 1.0 wt % magnesium stearate.

24. The composition of claim 23, wherein (a) ranges from about 15.5 to about 18.7 wt %.

25. The composition of claim 23, wherein (a) is gepirone as a free base.

26. The composition of claim 23, wherein (a) is a pharmaceutically acceptable salt form of gepirone.

27. The composition of claim 26, wherein the pharmaceutically acceptable salt form of gepirone is gepirone hydrochloride.

28. The composition of claim 23, wherein (a) is a bioactive metabolite of gepirone or pharmaceutically acceptable salt thereof.

29. The composition of claim 28, wherein said bioactive metabolite of gepirone is selected from the group consisting of 3′-OH gepirone, 3′,5-dihydroxy gepirone, and 5-OH gepirone.

30. The composition of claim 23, wherein (c) ranges from 0 to about 0.3 wt %.

31. The composition of claim 23, wherein (c) is one or more iron oxides.

32. The composition of claim 23, wherein (e) ranges from about 0.42 to about 0.47 wt %.

33. A method of treating depression in a patient in need thereof, comprising administering to said patient an effective amount of the composition of claim 23.

34. The method of claim 33, wherein said patient is human.

35. The method of claim 33, wherein said patient further suffers from generalized anxiety.

36. The method of claim 33, wherein said effective amount ranges from 0.01 to 40 mg/kg body weight per day.

37. The composition of claim 23, wherein said composition is in a form of a tablet.

38. The composition of claim 37, wherein the total weight of said tablet ranges from 350 to 450 mg.

39. The composition of claim 38, wherein the weight of (a) in said table ranges from 60 to 80 mg.

40. The composition of claim 37, wherein said tablet has an ovoid-rectangular shape with biconvex faces.

41. The composition of claim 40, wherein said tablet has an overall dimension of 0.400±0.05 inches by 0.325±0.05 inches with a thickness of 0.240±0.025 inches.

42. The composition of claim 37, wherein said tablet has an ovoid-rectangular shape with flat faces.

43. The composition of claim 42, wherein said tablet has an overall dimension of 0.400±0.05 inches by 0.325±0.05 inches with a thickness of 0.195±0.025 inches.

44. A method of treating depression in a patient in need thereof, comprising administering to said patient an effective amount of the composition of claim 37.

45. The method of claim 44, wherein said patient is human.

46. The method of claim 44, wherein said patient further suffers from generalized anxiety.

47. The method of claim 44, wherein said effective amount ranges from 0.01 to 40 mg/kg body weight per day.

48. A method of making a tablet of claim 37 comprising:
(i) admixing all or a part of components (a) through (f),
(ii) blending the mixture obtained in (i),
(iii) adding a part of or the remainder of components (a) through (f),
(iv) blending the mixture obtained in (iii), if any components where added during (iii),
(v) slugging the blend from (iv),
(vi) milling the slug obtained in (v),
(vii) adding the remainder of component (f); if part reserved during (i) and (iv),
(viii) blending the mixture from (vii);
(ix) compressing the blend from (viii) to form tablet.

49. A method of making a tablet of claim 37 comprising:
i) admixing all of component (a), all of component (c), all of component (e), and 20% of the total concentration of component (b);
ii) blending the mixture from (i);
iii) adding to the blend from (ii) all of component (d), half of the total concentration of component (f), and the remaining 80% of the total concentration of component (b);
iv) blending the mixture from (iii);
v) slugging the blend from (iv);
vi) milling the slug from (v);
vii) adding the remaining 50% of component (f) to the milled slug from (vi);
viii) blending the mixture from (vii);
ix) compressing the blend from (viii) into the final desired form.

50. The composition of claim 37, comprising:
(a) about 15.6 wt % of gepirone hydrochloride
(b) about 75.3 wt % hydroxypropylmethylcellulose,
(c) about 0.31 wt % yellow ferric oxide,
(d) about 8.0 wt % microcrystalline cellulose,
(e) about 0.42 wt % colloidal silica, and
(f) about 0.31 wt % magnesium stearate.

51. The composition of claim 50, wherein the total weight of said tablet is 385 mg.

52. The composition of claim 51, wherein the weight of (a) in said table is 60 mg.

53. The composition of claim 50, wherein said tablet has an ovoid-rectangular shape with biconvex faces.

54. The composition of claim 53, wherein said tablet has an overall dimension of 0.400±0.05 inches by 0.325±0.05 inches with a thickness of 0.240±0.025 inches.

55. The composition of claim 50, wherein said tablet has an ovoid-rectangular shape with flat faces.

56. The composition of claim 55, wherein said tablet has an overall dimension of 0.400±0.05 inches by 0.325±0.05 inches with a thickness of 0.195±0.025 inches.

57. A method of treating depression in a patient in need thereof, comprising administering to said patient an effective amount of the composition of claim 50.

58. The method of claim 57, wherein said patient is human.

59. The method of claim 57, wherein said patient further suffers from generalized anxiety.

60. The method of claim 57, wherein said effective amount ranges from 0.01 to 40 mg/kg body weight per day.

61. The composition of claim 37, consisting essentially of:
(a) about 15.6 wt % of gepirone hydrochloride
(b) about 75.3 wt % hydroxypropylmethylcellulose,
(c) about 0.31 wt % yellow ferric oxide,
(d) about 8.0 wt % microcrystalline cellulose,
(e) about 0.42 wt % colloidal silica, and
(f) about 0.31 wt % magnesium stearate.

62. The composition of claim 61, wherein the total weight of said tablet is 385 mg.

63. The composition of claim 62, wherein the weight of (a) in said table is 60 mg.

64. The composition of claim 61, wherein said tablet has an ovoid-rectangular shape with biconvex faces.

65. The composition of claim 64, wherein said tablet has an overall dimension of 0.400±0.05 inches by 0.325±0.05 inches with a thickness of 0.240±0.025 inches.

66. The composition of claim 61, wherein said tablet has an ovoid-rectangular shape with flat faces.

67. The composition of claim 66, wherein said tablet has an overall dimension of 0.400±0.05 inches by 0.325±0.05 inches with a thickness of 0.195±0.025 inches.

68. A method of treating depression in a patient in need thereof, comprising administering to said patient an effective amount of the composition of claim 61.

69. The method of claim 68, wherein said patient is human.

70. The method of claim 68, wherein said patient further suffers from generalized anxiety.

71. The method of claim 68, wherein said effective amount ranges from 0.01 to 40 mg/kg body weight per day.

72. The composition of claim 37, comprising:
(a) about 19.5 wt % of gepirone hydrochloride
(b) about 70.7 wt % hydroxypropylmethylcellulose,
(c-1) about 0.24 wt % yellow ferric oxide,
(c-2) about 0.61 wt % red ferrid oxide
(d) about 8.2 wt % microcrystalline cellulose,
(e) about 0.39 wt % colloidal silica, and
(f) about 0.29 wt % magnesium stearate.

73. The composition of claim 72, wherein the total weight of said tablet is 410 mg.

74. The composition of claim 73, wherein the weight of (a) in said table is 80 mg.

75. The composition of claim 72, wherein said tablet has an ovoid-rectangular shape with biconvex faces.

76. The composition of claim 75, wherein said tablet has an overall dimension of 0.400±0.05 inches by 0.325±0.05 inches with a thickness of 0.240±0.025 inches.

77. The composition of claim 72, wherein said tablet has an ovoid-rectangular shape with flat faces.

78. The composition of claim 77, wherein said tablet has an overall dimension of 0.400±0.05 inches by 0.325±0.05 inches with a thickness of 0.195±0.025 inches.

79. A method of treating depression in a patient in need thereof, comprising administering to said patient an effective amount of the composition of claim 72.

80. The method of claim 79, wherein said patient is human.

81. The method of claim 79, wherein said patient further suffers from generalized anxiety.

82. The method of claim 79, wherein said effective amount ranges from 0.01 to 40 mg/kg body weight per day.

83. The composition of claim 37, consisting essentially of:
(a) about 19.5 wt % of gepirone hydrochloride
(b) about 70.7 wt % hydroxypropylmethylcellulose,
(c-1) about 0.24 wt % yellow ferric oxide,
(c-2) about 0.61 wt % red ferrid oxide
(d) about 8.2 wt % microcrystalline cellulose,
(e) about 0.39 wt % colloidal silica, and
(f) about 0.29 wt % magnesium stearate.

84. The composition of claim 83, wherein the total weight of said tablet is 410 mg.

85. The composition of claim 84, wherein the weight of (a) in said table is 80 mg.

86. The composition of claim 83, wherein said tablet has an ovoid-rectangular shape with biconvex faces.

87. The composition of claim 86, wherein said tablet has an overall dimension of 0.400±0.05 inches by 0.325±0.05 inches with a thickness of 0.240±0.025 inches.

88. The composition of claim 83, wherein said tablet has an ovoid-rectangular shape with flat faces.

89. The composition of claim 88, wherein said tablet has an overall dimension of 0.400±0.05 inches by 0.325±0.05 inches with a thickness of 0.195±0.025 inches.

90. A method of treating depression in a patient in need thereof, comprising administering to said patient an effective amount of the composition of claim 83.

91. The method of claim 90, wherein said patient is human.

92. The method of claim 90, wherein said patient further suffers from generalized anxiety.

93. The method of claim 90, wherein said effective amount ranges from 0.01 to 40 mg/kg body weight per day.
